(19) United States
(12) Patent Application Publication
(10) Pub. No.: US 2007/0243244 A1
(43) Pub. Date: Oct. 18, 2007

(54) Methods of treating gastrointestinal tract infections with tigecycline

(76) Inventors: Syed M. Shah, East Hanover, NJ (US); Mahdi B. Fawzi, Morristown, NJ (US)

Correspondence Address: WYETH/FINNEGAN HENDERSON, LLP, 901 NEW YORK AVENUE, NW, WASHINGTON, DC, 20001-4413, US

(21) Appl. No.: 11/642,522

(22) Filed: Dec. 21, 2006

Related U.S. Application Data
(60) Provisional application No. 60/753,161, filed on Dec. 22, 2005.

(51) Int. Cl. A61K 31/65; A61K 9/40; A61K 9/50; A61K 9/56

(52) U.S. Cl. 424/456; 424/463; 514/152

(57) ABSTRACT

Disclosed herein are methods of treating at least one bacterial infection, such as lower gastrointestinal infections, comprising orally administering a pharmaceutical composition comprising tigecycline. The composition can take solid or liquid forms, such as solutions, dispersions, or solid forms comprising tigecycline having at least one enteric coating.

### CROSS-REFERENCE TO RELATED APPLICATIONS

This application claims the benefit of U.S. Provisional Application No. 60/753,161, filed Dec. 22, 2005 which is incorporated herein by reference in its entirety.

In one embodiment, this invention relates to methods of treating gastrointestinal tract infections with oral formulations comprising tigecycline.

Tigecycline is a glycylcycline antibiotic, i.e., a t-butylglycyl substituted naphthacenecarboxamide free base, and an analog of the semisynthetic tetracycline, minocycline.

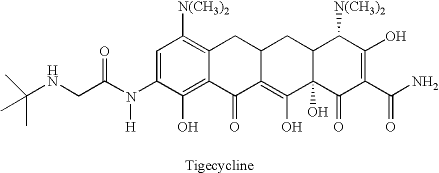

Tetracyclines such as chlortetracycline hydrochloride (Aureomycin) and oxytetracycline (Terramycin) are safe and have been used therapeutically as broad-spectrum antibiotics since 1948. However, the emergence of resistance to these antibiotics had limited their continued widespread usage. Tigecycline was thus developed as an agent to potentially restore therapeutic utility to tetracyclines by overcoming tetracycline resistance mechanisms. Tigecycline may also provide activity against emerging multi-drug resistant pathogens. Glycylcyclines, including tigecycline, are active against many antibiotic-resistant gram-positive pathogenic bacteria, such as methicillin-resistant Staphylococcus aureus, penicillin-resistant Streptococcus pneumoniae, and vancomycin-resistant enterococci (Weiss et al., 1995; Fraise et al., 1995). Tigecycline is also active against bacterial strains carrying the two major forms of tetracycline resistance, efflux and ribosomal protection (Schnappinger and Hillen, 1995).

There have been investigations in the treatment of infections in the gastrointestinal tract. For example, Vancocine® is an oral capsule form of the I.V. drug vancomycin, which is used to treat infections of the colon and the intestine, including those caused by strains of the Staphylococcus bacterium or Clostridium Difficile that do not respond to more common antibiotics. C. difficile is a bacterium, which under certain circumstances, typically after antibiotic therapy, can colonize in the lower gastrointestinal tract where it may produce toxins that can cause inflammation of the colon and diarrhea, and possibly associated complications of disease. Advanced age, gastrointestinal surgery/manipulation, long length of stay in healthcare settings, underlying illnesses, and immunocompromising conditions can be associated with increased risk of disease. According to the CDC, there are approximately 3,000,000 cases of antibiotic associated diarrhea per year, of which 15 to 25 percent are caused by C. difficile.

Vancomycin is not absorbed in the G.I. tract, when dosed orally. Moreover, Vancocin® has relatively low activity (M.I.C.) against Clostridium Difficile, which may result in the need for high doses of oral vancomycin (125 mg or 250 mg). High doses may also have the potential of producing undesirable side effects.

Although an intravenous formulation of tigecycline has been prepared, simple oral immediate release prototypes containing tigecycline have resulted in poor bioavailability in animals. (Petersen et al., Antimicrobial Agents and Chemotherapy, Apr. 1999, Vol. 43, No. 4 p. 738-744.) However, the effectiveness of such oral formulations have not been tested against Clostridium Difficile conditions.

Tigecycline is very soluble in water with solubility greater than 295 mg/mL over the entire pH range of 1 to 14. However, cell monolayer permeability studies of tigecycline (1 mM in ethanol and buffer, pH 6 to 6.4) show a low value of 0.4 nm s⁻¹, suggesting a low GI permeability, which is consistent with the low oral bioavailability found in animals.

Accordingly, there remains a need to develop a method for treating gastrointestinal tract infections with tigecycline.

### BRIEF DESCRIPTION OF THE DRAWINGS

FIG. 1 is a plot of percent release of tigecycline (y-axis) versus time (x-axis, min);

FIG. 2 shows the analytical performance of tigecycline in monkey plasma, low QC (quality control)—300 ng/mL as a plot of tigecycline plasma concentration (y-axis) vs. curve number (x-axis);

FIG. 3 shows the analytical performance of tigecycline in monkey plasma, mid QC A—663 ng/mL as a plot of tigecycline plasma concentration (y-axis) vs. curve number (x-axis);

FIG. 4 shows the analytical performance of tigecycline in monkey plasma, mid QC B—556 ng/mL as a plot of tigecycline plasma concentration (y-axis) vs. curve number (x-axis);

FIG. 5 shows the analytical performance of tigecycline in monkey plasma, high QC—3000 ng/mL as a plot of tigecycline plasma concentration (y-axis) vs. curve number (x-axis);

FIG. 6 is a plot of plasma concentration (y-axis) vs. time (x-axis) profile of tigecycline in monkeys after a single intravenous dose of 5 mg/kg;

FIG. 7 is a plot of tigecycline plasma concentration (y-axis) vs. curve number (x-axis), showing the analytical performance of tigecycline assay in monkey plasma: low QC (quality control)—30 ng/mL;

FIG. 8 is a plot of tigecycline plasma concentration (y-axis) vs. curve number (x-axis), showing the analytical performance of tigecycline assay in monkey plasma: middle QC—300 ng/mL;

FIG. 9 is a plot of tigecycline plasma concentration (y-axis) vs. curve number (x-axis), showing the analytical performance of tigecycline assay in monkey plasma: high QC—800 ng/mL; and

FIG. 10 is a plot of plasma concentration of tigecycline (ng/ml, y-axis) vs. time (h, x-axis) after a single oral dose (100 mg encapsulated microparticulate capsule) in fasted male cynomolgus monkey.

One embodiment of the present invention provides a method of treating at least one bacterial infection, comprising:

orally administering to a subject in need thereof a pharmaceutical composition comprising a therapeutically effective amount of tigecycline.

In one embodiment, the at least one bacterial infection is a gastrointestinal (GI) infection, i.e., the infection occurs in.the gastrointestinal tract. The gastrointestinal tract includes the upper and lower GI tract. The upper GI tract includes the stomach and esophagus. In one embodiment, “lower gastrointestinal tract” as used herein refers to the ileum and large intestine. “Ileum” as used herein refers to a third part of the small intestine that continues to the duodenum and jejunum. “Large intestine” as used herein comprises the cecum, colon and rectum. “Cecum” refers to a blind sack (cul-de-sac) starting from the large intestine and in one end of which the ileum opens.

In one embodiment, the at least one bacterial infection is caused by anaerobic bacteria.

In one embodiment, the at least one bacterial infection is caused by Clostridium difficile. C. difficile is a bacterium, which under certain circumstances can colonize in the lower gastrointestinal tract where it may produce toxins that can cause inflammation of the colon and diarrhea. In one embodiment, the treatment can result in treatment of the infection and/or associated complications of disease. Moreover, an emerging genotype of C. difficile produces toxin levels that are 16-23 times higher than in previously identified strains.

Although previous studies (Petersen et al.) have shown low blood bioavailability of tigecycline when a simple oral immediate release prototype was administered, tigecylcine's high bioactivity (e.g., when compared to vancomycin) against bacterial infections, such as C. difficile, can nonetheless result in an effective treatment. In one embodiment, when treating gastrointestinal tract infections, the low blood bioavailability indicates that the bioavailability in the GI tract is high, i.e., the majority of the formulation is present in the stomach.

Another embodiment provides a method of treating antibiotic associated pseudomembranous colitis caused by C. difficile and enterocolitis caused by S. aureus and associated methicillin resistant strains comprising:

orally administering to a subject in need thereof a pharmaceutical composition comprising a therapeutically effective amount of tigecycline.

In one embodiment, “orally administering” comprises allowing the patient to swallow the pharmaceutical composition. In another embodiment, the orally administering is performed via a nasal-gastric tube for delivery to the large intestine.

“Pharmaceutical composition” as used herein refers to a medicinal composition in solid or liquid form. The pharmaceutical composition may contain at least one pharmaceutically acceptable carrier.

In one embodiment, the composition further comprises at least one inert, pharmaceutically-acceptable excipient or carrier. “Pharmaceutically acceptable excipient” as used herein refers to pharmaceutical carriers or vehicles suitable for administration of tigecycline including any such carriers known to those skilled in the art to be suitable for oral administration.

In one embodiment, the oral formulation does not release a substantial amount of tigecycline in the stomach and a substantial release occurs when the formulation reaches the gastrointestinal tract, such as the lower gastrointestinal tract. In one embodiment, the pharmaceutical composition comprises tigecycline having an enteric coating. In one embodiment, “having an enteric coating” refers to surrounding a bulk of tigecycline. In another embodiment, the enteric coating surrounds substantially each Tigecycline particle. “Coating” can comprise either a coating or subcoating. “Coating,” or “surrounds” as used herein, may range, for example, from at least partially coating or surrounding up to and including a complete coating or surrounding. In one embodiment, coating or surrounding refers to substantially coating, such as 90%, 95%, and 99% coating by weight. In one embodiment, the enteric coating may be sufficiently uniform to confer physical stability to the tigecycline, e.g., by preventing degradation by any method disclosed herein.

In one embodiment, an “enteric coating” can allow at least a substantial portion of a formulation to pass through the stomach and disintegrate in the intestines. Exemplary materials for the preparation of enteric coatings include, but are not limited to dimethylaminoethyl methacrylatemethylacrylate acid ester copolymer, anionic acrylic resins such as methacrylic acid/methyl acrylate copolymer and methacrylic acid/ethyl acrylate copolymer, ethylacrylate-methylmethacrylate copolymer, hydroxypropylmethylcellulose acetate succinate (HPMCAS), hydroxypropylmethylcellulose phthalate (HPMCP), cellulose acetate phthalate (CAP), carboxymethylcellulose acetate phthalate (CMCAP), hydroxypropylmethylcellulose, hydroxyethylcellulose, methylhydroxyethylcellulose, sodium carboxymethylcellulose, hydroxypropylcellulose, polyvinyl pyrrolidone, shellac, methylcellulose, and ethylcellulose, and blends and copolymers thereof.

In one embodiment, the enteric coating may be formed by methods known in the art for forming polymeric films.

In one embodiment, the composition further comprises a seal coat. In one embodiment, the seal coat is positioned underneath the enteric coat. In another embodiment, the composition can contain at least one additional seal coat that overcoats the enteric coat, which in turn overcoats a first seal coat. In one embodiment, the seal coat comprises any material suitable for forming enteric coatings, such as hydroxypropyl cellulose, polyvinyl pyrrolidone, sodium carboxymethylcellulose, and hypromellose, or any other enteric coating material disclosed herein.

In one embodiment, the at least one enteric coating can protect tigecycline from substantial degradation. Tigecycline may have at least two degradation mechanisms. At low pH, epimerization of the dimethylamino group at 4-position has been identified as a major degradation route. At pH higher than 7.4, the degradation mechanism shifts to oxidation, as the phenolic groups can become deprotonated. Tigecycline can, for example, be stabilized in both solid and solution states by eliminating oxygen. Once oxygen is eliminated, the pH of optimum stability shifts from 4.5 to 8 where epimerization is at its minimum.

In one embodiment, the composition further comprises at least one chelating agent. Calcium binds to tetracyclines, which reduce its water solubility. There may be a 30 to 40% loss of tigecycline due to precipitation of the calcium complex at pH 7.4. Thus, calcium binding and subsequent precipitation of the calcium/tigecycline salt may be at least partially responsible for low oral bioavailability. Exemplary chelating agents include ethylenediaminetetraacetic acid (EDTA), O,O′-bis(2-aminoethyl)ethyleneglycol-N,N,N′,N′-tetraacetic acid (EGTA), citrates, and tartrates.

In one embodiment, the composition further comprises at least one base. In one embodiment, the at least one base provides the composition with a microenvironment having a pH ranging from 4 to 8.5 when released, such as a pH ranging from 7.8 to 8.5 when released. In one embodiment, the pH of the microenvironment refers to the pH of the area immediately surrounding the composition. In another embodiment, the microenvironment refers to the area inside the seal coat. Exemplary bases include, but are not limited to, phosphates, such as at least one sodium phosphate, carbonates such as sodium and potassium carbonate, bicarbonates, such as sodium and potassium bicarbonate, citrates, such as sodium citrate, and tartrates.

Additionally, in some embodiments, buffer species can negatively affect the stability of tigecycline. In one embodiment, the at least one base may be capable of countering the effects of such buffer species.

In one embodiment, the composition further comprises at least one biopolymer. For example, in embodiments where the composition is used to treat infections in the GI tract, such as the inner or lower GI tract, the at least one biopolymer can act as an adhesive to the inner GI tract and therefore allow for enhanced absorption of tigecycline. Exemplary biopolymers include, but are not limited to, hypromellose and xanthan gum, and carbomer.

Suitable excipients include, for example, (a) fillers or extenders such as starches, lactose, sucrose, glucose, mannitol, and silicic acid; (b) binders such as cellulose and cellulose derivatives (such as hydroxypropylmethylcellulose, hydroxypropylcellulose, and carboxymethylcellulose), alginates, gelatin, polyvinylpyrrolidone, sucrose, and acacia; (c) humectants such as glycerol; (d) disintegrating agents such as sodium starch glycolate, croscarmellose, agar-agar, calcium carbonate, potato or tapioca starch, alginic acid, certain silicates, and sodium carbonate; (e) solution retarding agents such as paraffin; (f) absorption accelerators such as quaternary ammonium compounds; (g) wetting agents, such as cetyl alcohol and glycerol monostearate, fatty acid esters of sorbitan, poloxamers, and polyethylene glycols; (h) absorbents such as kaolin and bentonite clay; (i) lubricants such as talc, calcium stearate, magnesium stearate, solid polyethylene glycols, sodium lauryl sulfate, and mixtures thereof; and (j) glidants (antiadherents) such as talc, and silicone dioxide. Other suitable excipients include, for example, sodium citrate or dicalcium phosphate. The dosage forms may also comprise buffering agents.

Oral formulations may also employ fillers in soft and hard-filled gelatin capsules using such excipients as lactose or milk sugar as well as high molecular weight polyethylene glycols.

In one embodiment, the pharmaceutical composition is in liquid form. Such compositions may comprise pharmaceutically-acceptable aqueous or nonaqueous solutions, dispersions, suspensions or emulsions as well as sterile powders and/or lyophilized powders for reconstitution into sterile solutions or dispersions just prior to use. Examples of suitable aqueous and nonaqueous carriers, diluents, solvents or vehicles include water, ethanol, polyols (such as glycerol, propylene glycol, and polyethylene glycol), and suitable mixtures thereof, vegetable oils (such as olive oil), and organic esters such as ethyl oleate. Proper fluidity can be maintained, for example, by the use of coating materials such as lecithin, by the maintenance of the required particle size in the case of dispersions, and by the use of surfactants.

In one embodiment, the liquid form is a solution or suspension having a pH of less than 7.5.

In one embodiment, the liquid form is provided in vials or other suitable containers.

These compositions may also contain adjuvants such as preservatives, wetting agents, emulsifying agents, and dispersing agents. They may also contain taggants or other anti-counterfeiting agents, which are well known in the art. Prevention of the action of microorganisms may be ensured by the inclusion of various antibacterial and antifungal agents, for example, paraben, chlorobutanol, and phenol sorbic acid. It may also be desirable to include isotonic agents such as sugars, and sodium chloride. Prolonged absorption of the liquid pharmaceutical form may be brought about by the inclusion of agents, which delay absorption such as aluminum monostearate and gelatin.

Liquid dosage forms include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers such as cyclodextrins, ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols, and fatty acid esters of sorbitan, and mixtures thereof.

Suspensions, in addition to the active compounds, may contain at least one suspending agent such as, for example, xanthan gum, guar gum, gum arabic, hydroxypropylmethylcellulose, ethoxylated isostearyl alcohols, polyoxyethylene sorbitol and sorbitan esters, cellulose or cellulose derivatives (for example microcrystalline cellulose), aluminum metahydroxide, bentonite, agar agar, and tragacanth, and mixtures thereof.

The pharmaceutical compositions may optionally contain opacifying agents and colorants. They may also be in a form capable of controlled or sustained release. Examples of embedding compositions that can be used for such purposes include polymeric substances and waxes.

Where the composition is a suspension containing powdered tigecycline, the suspension can further comprise, for example, from about 0.05% to 5% of suspending agent by weight, syrups containing, for example, from about 10% to 50% of sugar by weight, and elixirs containing, for example, from about 20% to 50% ethanol by weight.

The pharmaceutical compositions disclosed herein may contain, for example, an amount ranging from about 25% to about 90% of the active ingredient by weight relative to the total weight of the composition, or from about 5% and 60% by weight.

The tigecycline can be provided as a pharmaceutically acceptable salt. The terms “pharmaceutically acceptable salt” can refer to acid addition salts or base addition salts of the compounds in the present disclosure. A pharmaceutically acceptable salt is any salt which retains the activity of the parent compound and does not impart any deleterious or undesirable effect on the subject to whom it is administered and in the context in which it is administered. Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, cilexetil, citric, edetic, edisylic, estolic, esyl, esylic, formic, fumaric, gluceptic, gluconic, glutamic, glycolic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic, toluenesulfonic, and the like. Pharmaceutically acceptable salts may be derived from amino acids, including but not limited to cysteine. Other acceptable salts may be found, for example, in Stahl et al., Pharmaceutical Salts: Properties, Selection, and Use, Wiley-VCH; 1st edition (Jun. 15, 2002).

Another embodiment provides a method of preparing a pharmaceutical composition comprising coating a tigecycline with at least one enteric coating. The coating can be performed using any known process in the art, such as by introducing the tigecycline into a fluid bed processor (or other coating device, such as a pan coater) containing the enteric coating material. Prior to its introduction into the coating device,. the tigecycline can be combined with one or more of at least one base/buffer, at least one chelating agent, at least one biopolymer, and other ingredients suitable for the oral formulation.

In one embodiment, “therapeutically effective amount” refers to that amount of a compound that results in prevention or amelioration of symptoms in a patient or a desired biological outcome, e.g., improved clinical signs, delayed onset of disease, reduced/elevated levels of lymphocytes and/or antibodies, etc. The effective amount can be determined by one of ordinary skill in the art. The selected dosage level can depend upon the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved.

In one embodiment, the subject treated can be a mammal, such as a human. In one embodiment, the subject is suspected of having a bacterial infection, e.g., shows at least one symptom associated with the infection. In another embodiment, the subject is one susceptible to having the bacterial infection, for example, a subject genetically disposed to having the disease.

“Treating” as used herein refers to both therapeutic treatment and prophylactic/preventative measures. Those in need of treatment may include individuals already having a particular medical disease as well as those at risk for the disease (i.e., those who are likely to ultimately acquire the disorder). A therapeutic method results in the prevention or amelioration of symptoms or an otherwise desired biological outcome and may be evaluated by improved clinical signs, delayed onset of disease, reduced/elevated levels of lymphocytes and/or antibodies, etc.

Actual dosage levels of tigecycline in the pharmaceutical compositions of this invention may be varied so as to obtain the therapeutically effective amount necessary to achieve the desired therapeutic response for a particular patient.

Generally dosage levels of about 0.1 μg/kg to about 50 mg/kg, such as a level ranging from about 5 to about 20 mg of active compound per kilogram of body weight per day, can be administered topically, orally or intravenously to a mammalian patient. Other dosage levels range from about 1 μg/kg to about 20 mg/kg, from about 1 μg/kg to about 10 mg/kg, from about 1 μg /kg to about 1 mg/kg, from 10 μg/kg to 1 mg/kg, from 10 μg/kg to 100 μg/kg, from 100 μg to 1 mg/kg, and from about 500 μg/kg to about 5 mg/kg per day. If desired, the effective daily dose may be divided into multiple doses for purposes of administration, e.g., two to four separate doses per day. In one embodiment, the pharmaceutical composition can be administered once or twice per day.

In one embodiment, the tigecycline is multi-particulate. As used herein, “multi-particulate tigecycline” refers to a collection of tigecycline particles. In one embodiment, the multi-particulate tigecycline has a mean particle size ranging from 0.3 mm to 1.5 mm. The multi-particulate tigecycline can be provided as a powder, or provided as a capsule encased within a shell, or any other dosage form as described herein.

In one embodiment, dosage forms for oral administration include, but are not limited to, capsules, tablets, pills, powders (e.g., dispersible powders, suspensions containing such powders), dragees, granules, and lyophilized cakes and powders. Such forms may include forms that dissolve or disintegrate quickly in the oral environment. In another embodiment, the oral dosage form slows the dissolution of the drug immediately following oral administration and allows a substantial portion of the dissolution to occur in the GI tract, such as the lower GI tract. In one embodiment, the dosage form (e.g., powders, cakes) is provided in vials or other suitable containers.

In one embodiment, the pharmaceutical composition is a saline solution containing tigecycline.

In another embodiment, the composition is a dispersion comprising tigecycline.

In one embodiment, the pharmaceutical composition comprises a compressed tablet containing tigecycline in an amount ranging from 100 mg to 300 mg.

In one embodiment, the pharmaceutical composition comprises enteric coated multi-particulate pellets incorporated into a hard gelatin capsule, and each pellet comprising tigecycline and microcrystalline cellulose, and a combination of one or more of the following: at least one base/buffer (e.g., at least one sodium phosphate), at least one chelating agent (e.g., EDTA), and at least one biopolymer (e.g., xanthan gum).

In one embodiment, the pharmaceutical composition comprises an enteric coated tablet comprising tigecycline and microcrystalline cellulose, and further comprises one or more of the following: at least one base/buffer (e.g., at least one sodium phosphate), at least one chelating agent (e.g., EDTA), and at least one biopolymer (e.g., xanthan gum).

In one embodiment, the pharmaceutical composition comprises multi-particulate pellets incorporated into an enteric coated soft gelatin capsule, and each pellet comprising tigecycline and microcrystalline cellulose, and one or more of the following: at least one base/buffer (e.g., at least one sodium phosphate), at least one chelating agent (e.g., EDTA), and at least one biopolymer (e.g., xanthan gum).

In one embodiment, the pharmaceutical composition comprises an enteric coated soft liquid gel capsule, and further comprising a non-aqueous solution of tigecycline, and one or more of the following: at least one base/buffer (e.g., at least one sodium phosphate), at least one chelating agent (e.g., EDTA), and at least one biopolymer (e.g., xanthan gum).

In one embodiment, the pharmaceutical composition comprises a capsule or bi-layer tablet comprising both an immediate release portion and an extended release portion. In one embodiment, “extended release” involves release of substantially all of the tigecycline over a time period of at least 4 hours, such as a time period of at least 6 hours, at least 12 hours, at least 24 hours, or at least 48 hours.

In one embodiment, the pharmaceutical composition comprises tigecycline in solid form, the composition further comprising lactose and at least one acidifying agent. The at least one acidifying agent can include any of the organic or inorganic acids disclosed herein. In one embodiment, the at least one acidifying agent is HCl.

In one embodiment, the pharmaceutical composition comprises a suspension, wherein the suspension comprises granules and at least one suspending agent. Exemplary suspending agents are chosen from xanthan gum, guar gum, gum arabic, and hydroxypropylmethylcellulose, and any other suspending agent disclosed herein.

In one embodiment, the pharmaceutical composition may be used as a treatment against drug-resistant bacteria. For example, it may be active against methicillin-resistant Staphylococcus aureus, penicillin-resistant Streptococcus pneumoniae, vancomycin-resistant enterococci (D. J. Beidenbach et. al., Diagnostic Microbiology and Infectious Disease 40:173-177 (2001); H. W. Boucher et. al., Antimicrobial Agents & Chemotherapy 44:2225-2229 (2000); P. A. Bradford Clin. Microbiol. Newsleft. 26:163-168 (2004); D. Milatovic et. al., Antimicrob. Agents Chemother. 47:400-404 (2003); R. Patel et. al., Diagnostic Microbiology and Infectious Disease 38:177-179 (2000); P. J. Petersen et. al., Antimicrob. Agents Chemother. 46:2595-2601 (2002); and P. J. Petersen et. al., Antimicrob. Agents Chemother. 43:738-744 (1999), and against organisms carrying either of the two major forms of tetracycline resistance: efflux and ribosomal protection (C. Betriu et. al., Antimicrob. Agents Chemother. 48:323-325 (2004); T. Hirata et. al. Antimicrob. Agents Chemother. 48:2179-2184 (2004); and P. J. Petersen et. al., Antimicrob. Agents Chemother. 43:738-744 (1999).

In one embodiment, the pharmaceutical composition may be used in the treatment of many bacterial infections, such as complicated intra-abdominal infections (cIAI), complicated skin and skin structure infections (cSSSI), Community Acquired Pneumonia (CAP), and Hospital Acquired Pneumonia (HAP) indications, which may be caused by gram-negative and gram-positive pathogens, anaerobes, and both methicillin-susceptible and methicillin-resistant strains of Staphylococcus aureus (MSSA and MRSA). Additionally, the pharmaceutical composition may be used to treat or control bacterial infections in warm-blooded animals caused by bacteria having the TetM and TetK resistant determinants. Also, the pharmaceutical composition may be used to treat bone and joint infections, catheter-related Neutropenia, obstetrics and gynecological infections, or to treat other resistant pathogens, such as VRE, ESBL, enterics, rapid growing mycobacteria, and the like.

### EXAMPLES

### Example 1

In this Example, the dissolution behavior of enteric coated tigecycline granules in capsules was investigated in a solution of 0.1 N HCI, then in phosphate buffer pH 6.8 at 37° C. These conditions mimic the gastric system (0.1 N) and the lower intestinal tract (ph 6.8).

The formulation used is described in Example 3, below.

Gelatin capsules of enteric coated granules of 100 mg tigecycline were added to three separate vessels (Capsules 1, 2, and 3). The capsules were dissolved with a USP Apparatus 2 (paddles) at 100 rpm in 750 mL of 0.1 N HCl at 37° C. The dissolution was allowed to occur for 2 h, followed by addition of 250 mL of 0.2M Na₃PO₄. The pH of this mixture was adjusted to 6.8. Table I below lists the dissolution data.

TABLE I
Percent release of gelatin capsules of enteric coated
100 mg tigecycline granules

| Time (min) | Cap 1 | Cap 2 | Cap 3 |
|---|---|---|---|
| 0 | 0 | 0 | 0 |
| 30 | 11.14271 | 12.56791 | 11.28477 |
| 60 | 24.17531 | 25.30732 | 22.83157 |
| 90 | 30.8192 | 30.66811 | 29.8502 |
| 120 | 35.07275 | 35.47755 | 33.74161 |
| 125 | 39.30319 | 38.94879 | 37.98354 |
| 130 | 40.70022 | 40.81831 | 38.93004 |
| 135 | 42.28829 | 43.52615 | 41.04458 |
| 150 | 49.00615 | 47.11648 | 47.38426 |
| 180 | 52.64652 | 51.85096 | 51.09949 |
| 240 | 75.78954 | 70.31774 | 67.92135 |
| 300 | 79.53955 | 79.71117 | 81.44953 |

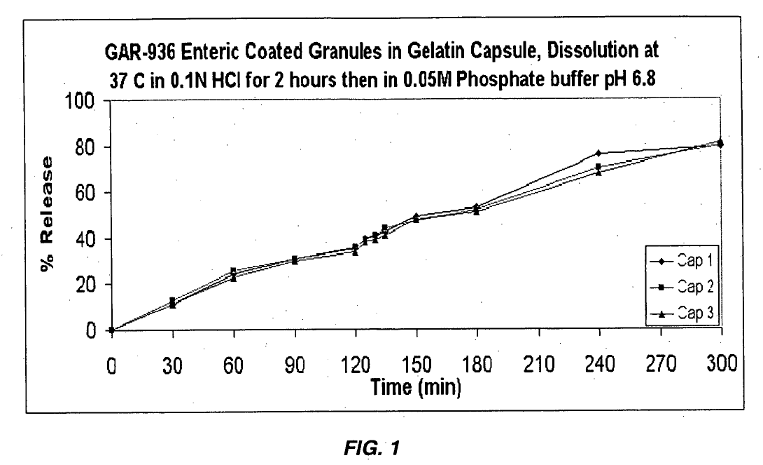

FIG. 1 is a plot of the data of Table I of percent release (x-axis) versus time (min). The ratio of AUC to mg/ml is according to the equation y=16279x−58.773.

This Example demonstrates that the formulation releases substantially cycline at higher pH, e.g., after 2 hours.

### Example 2

This Example demonstrates the oral bioavailability of tigecycline in cynomolgus monkeys when administered as an oral formulation (gavage). The pharmacokinetics of tigecycline after single oral and intravenous administration are also presented in this Example.

Male monkeys were first administered an oral (gavage) dose of 15 mg/kg of tigecycline and then an intravenous dose of 5 mg/kg of tigecycline after a one-week wash-out period.

### Materials and Methods

Study Design

Four male cynomolgus monkeys were used in the study. In a first dosing period, each monkey was administered a single 15 mg/kg oral (gavage) dose of tigecycline in 0.9% saline. The dosing volume was 10 mL/kg. Blood samples (2 mL per sample) were obtained prior to dosing (0 hr) and at 0.5, 1, 2, 4, 6, 8, 12, 24, 32 and 48 hr after the oral dose. After a one-week washout period, each monkey was administered a single 5 mg/kg intravenous dose of tigecycline in 0.9% saline. Blood samples (2 mL) were obtained pre-dose (0 hr) and at 5 mm., 0.5, 1, 2, 4, 6, 8, 12, 24, 32 and 48 hr post-dose. Blood samples were collected using a stainless steel needle and vacutainer tube containing sodium heparin as the anticoagulant. Blood samples were placed on ice after collection and centrifuged at approximately 4° C. Plasma samples was separated, frozen and stored at approximately −70° C. prior to analysis.

Quantitation of Tigecycline in Monkey Plasma

Tigecycline concentrations were determined using an HPLC method that was previously validated in rat and dog plasma, although this method was modified to be used in monkey plasma. In this method, tigecycline in 0.2 mL of monkey plasma samples was extracted by protein precipitation with acetonitrile and the precipitated proteins were separated by centrifugation. The supernatant was evaporated and the extract was reconstituted in 0.05N HCl for HPLC analysis. Regression analysis was performed on the calibration curve using a quadratic fit with a weighting factor of 1/(concentration)². By using 0.2 mL of monkey plasma sample, the assay limit of quantitation (LOQ) was 100 ng/mL and the curve range was between 100 and 6400 ng/mL.

Pharmacokinetic Calculations

Pharmacokinetic parameters were calculated using the pharmacokinetics analysis program WinNonlin, version 2.1 (Scientific Consulting Inc.) from the individual animal concentration vs. time profiles. This program analyzes data using a model-independent approach and the standard methods described by Gibaldi and Perrier (Gibaldi M, Perrier D., Pharmacokinetics, 2^{nd} ed., Marcel Dekker, Inc., NY, 1982). For the purpose of this analysis, no attempt was made to back extrapolate the concentration immediately after the IV bolus dose, rather the concentration at 0 hr (C₀, immediately after dosing) was assumed to be equal to the first measured concentration (at 5 minutes, C₅ₘᵢₙ). To determine the mean plasma drug concentrations, all values below the lower limit of quantitation (LOQ=100 ng/mL) were treated as zero. The terminal half-life (t_{1/2}) was determined by 0.693/λ, where λ is the terminal rate constant and is determined by a log-linear fitting of the terminal portion of the concentration-time curve. AUC₀₋₄ was calculated by AUC₀₋ₜ+Cₜ/λ, where AUC₀₋ₜ was the AUC from time 0 to t, the last quantifiable time point and Cₜ was the last quantifiable concentration. The area under the plasma concentration-time curve from time 0 to t (AUC₀₋ₜ) was calculated using the linear trapezoidal method. Systemic clearance (CL_{T}) after the iv dose was calculated using the formula of Dose/AUC₀₋₄. The volume of distribution at steady-state (Vdₛₛ) was calculated using the formula of MRTᵢᵥ×CL_{T}, where MRTᵢᵥ is the mean residence time after iv dosing and equals AUMC₀₋₄/AUC₀₋₄. For the oral dose, Cₘₐₓ and tₘₐₓ values were obtained by inspection of the concentration vs. time curves. Due to the paucity of quantifiable concentrations after oral administration, the AUC₀₋₄ could not be calculated.

Analytical Performance of the HPLC Method for Tigecycline in Monkey Plasma

Five analytical runs were performed for the analysis of samples. The back-calculated values of the calibration curves are presented in Table II. The CV of tigecycline calibartion standards were between 2.1 and 6.3% and the bias values ranged from −5.4 to 3.8%.

TABLE II
Analytical Performance of Tigecycline Assay in Monkey Plasma:
Back-Calculated Values of Tigecycline Calibration Standards

| | | Nominal concentration of tigecycline, ng/ML | | | | | | | | | |
|---|---|---|---|---|---|---|---|---|---|---|---|
| | No. | 100 | 200 | 400 | 500 | 800 | 1600 | 3200 | 4000 | 5000 | 6400 |
| | | Concentration of tigecycline found, ng/ML | | | | | | | | | |
| | 1 | 97.7 | 205 | 418 | 494 | 825 | 1604 | 3070 | 3861 | 4848 | 6709 |
| | 2 | 100 | 194 | 429 | NA | 763 | 1581 | 3284 | 3851 | 5158 | 6335 |
| | 3 | 100 | 202 | 416 | 478 | 724 | 1549 | 3510 | 4377 | 4829 | 6069 |
| | 4 | 103 | 189 | 404 | NA | 736 | 1652 | 3259 | 4300 | 5109 | 5996 |
| | 6 | 98.0 | 216 | 409 | 447 | 779 | 1512 | 3403 | 4297 | 5120 | 5968 |
| Mean | | 99.7 | 201 | 415 | 473 | 765 | 1580 | 3305 | 4137 | 5013 | 6215 |
| SD | | 2.12 | 10.4 | 9.52 | 23.9 | 39.8 | 53.3 | 165 | 259 | 160 | 312 |
| % CV | | 2.1 | 5.2 | 2.3 | 5.1 | 5.2 | 3.4 | 5.0 | 6.3 | 3.2 | 5.0 |
| % Bias | | −0.3 | 0.5 | 3.8 | −5.4 | −4.4 | −1.3 | 3.3 | 3.4 | 0.3 | −2.9 |
| n | | 5 | 5 | 5 | 3 | 5 | 5 | 5 | 5 | 5 | 5 |

NA: Not applicable

The calibration curve parameters are shown in Table III.

TABLE III
Analytical Performance of Tigecycline Assay in Monkey Plasma:
Calibration Curve Parameters

| | | 2^{nd} Order | 1^{st} Order | | |
|---|---|---|---|---|---|
| | Curve | Regression | Regression | | |
| | Number | Constant | Constant | Intercept | R² |
| | 1 | 0.0000 | 0.0000699 | −0.000908 | 0.9975 |
| | 2 | 0.0000 | 0.0000793 | −0.001800 | 0.9981 |
| | 3 | 0.0000 | 0.0000738 | −0.00262 | 0.9928 |
| | 4 | 0.0000 | 0.0000860 | −0.00348 | 0.9956 |
| | 6 | 0.0000 | 0.0000846 | −0.00274 | 0.9933 |
| Mean | | 0.0000 | 0.0000787 | −0.00231 | 0.9955 |
| SD | | 0.0000 | 0.0000069 | 0.000984 | 0.0024 |
| n | | 5 | 5 | 5 | 5 |

Regression analysis was performed with the following equation:
y=ax²+bx+c
where:
a=2^{nd} order regression line constant.
b=1^{st} order regression line constant.
c=Intercept.
y=Internal standard peak height ratio of tigecycline.
x=tigeycline concentration (ng/mL).

In all analytical runs, the coefficients of determination (R²) were >0.99. In all analytical runs, two replicates of low, mid-range and high QC samples were analyzed along with study samples. The low QC and the high QC have nominal concentrations of 300 and 3000 ng/mL, respectively. For the mid-range QC, the target nominal concentration was 900 ng/mL. Two separate batches of mid-range QC were prepared and both had concentrations below the target (ca. 600 ng/mL). The target concentrations of the mid-range QC batches were determined by analyzing four (batch A) or eight (batch B) replicates of each mid-range QC batch. Mid-range QC batch A (determined concentration of 663 ng/mL) was analyzed with curves 1 and 2. Mid-range QC batch B concentration of 556 ng/mL) was analyzed with curves 3, 4 and 6. The results of QC samples from all analytical runs are shown in Table IV.

TABLE IV
Analytical Performance of Tigecycline Assay in Monkey Plasma:
Results of QC Samples

| | | Low | Mid A | Mid B | High |
|---|---|---|---|---|---|
| | Curve | (300 | (663 | (556 | (3000 |
| | Number | ng/mL) | ng/mL) | ng/mL) | ng/mL) |
| | 1 | 288 | 729 | NA | 3310 |
| | | 319 | 762 | NA | 3281 |
| | 2 | 294 | 664 | NA | 3273 |
| | | 276 | 699 | NA | 3037 |
| | 3 | 293 | NA | 538 | 3211 |
| | | 295 | NA | 578 | 3302 |
| | 4 | 280 | NA | 632 | 2743 |
| | | 252 | NA | 650 | 2828 |
| | 6 | 273 | NA | 535 | 2628 |
| | | 395 | NA | 610 | 2579 |
| Mean | | 297 | 714 | 591 | 3019 |
| SD | | 38.8 | 41.8 | 48.2 | 297 |
| % CV | | 13.1 | 5.9 | 8.2 | 9.8 |
| % Bias | | −1.0 | 7.7 | 6.3 | 0.6 |
| n | | 10 | 4 | 6 | 10 |

NA: Not applicable; this QC batch was not analyzed with this run.

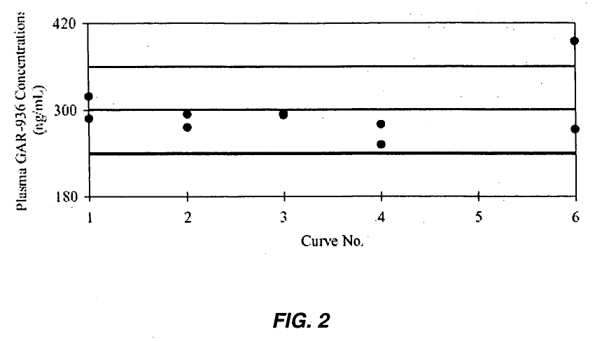

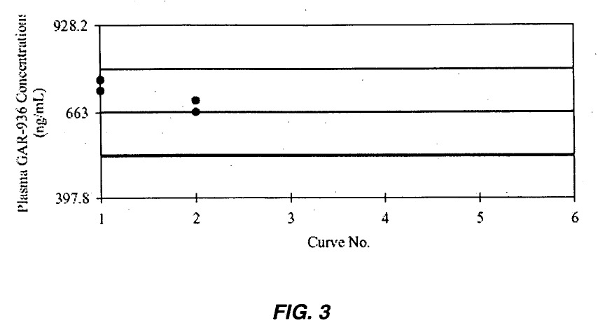

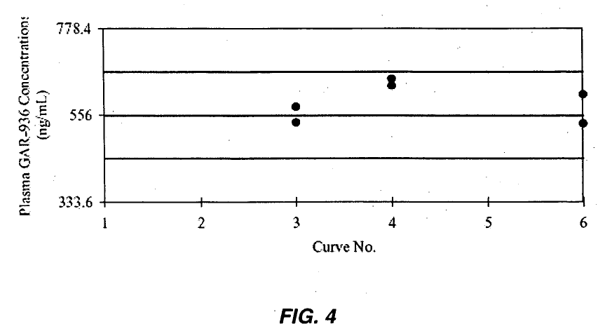

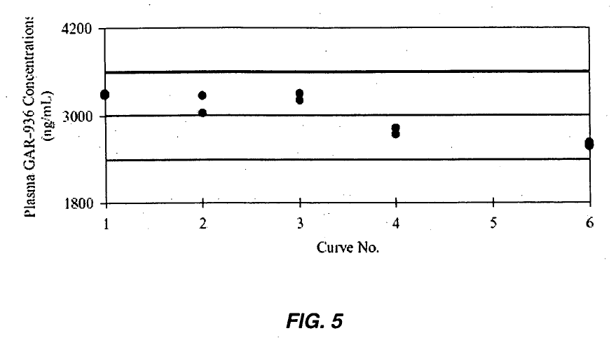

The CV of QC samples were between 5.9 and 13.1% and the biases were between −1.0 and 7.7%. The QC results are also depicted in QC charts and they are presented in FIGS. 2 to 5.

Pharmacokinetics of Tigecycline in Cynomolgus Monkeys

The concentrations of tigecycline after a single 15 mg/kg oral dose in monkeys are presented in Table V.

TABLE V
Plasma Concentrations (ng/mL) of Tigecycline in Monkeys After
a Single Oral (gavage) Dose of 15 mg/kg

| Animal No. | Hours | 0 | 0.5 | 1 | 2 | 4 | 6 | 8 | 12 | 24 | 32 | 48 |
|---|---|---|---|---|---|---|---|---|---|---|---|---|
| 1 | | <100 | <100 | 114 | 131 | <100 | <100 | <100 | <100 | <100 | <100 | <100 |
| 2 | | <100 | 101 | 128 | 191 | <100 | <100 | <100 | <100 | <100 | <100 | <100 |
| 3 | | <100 | 121 | 178 | <100 | <100 | <100 | <100 | <100 | <100 | <100 | <100 |
| 4 | | <100 | <100 | 105 | 150 | <100 | <100 | <100 | <100 | <100 | <100 | <100 |
| Mean | | 0 | 55.5 | 131 | 118 | 0 | 0 | 0 | 0 | 0 | 0 | 0 |
| SD | | 0 | 64.6 | 32.6 | 82.6 | 0 | 0 | 0 | 0 | 0 | 0 | 0 |
| n | | 4 | 4 | 4 | 4 | 4 | 4 | 4 | 4 | 4 | 4 | 4 |

The concentrations of tigecycline after a single 5 mg/kg iv dose are shown Table VI.

TABLE VI
Plasma Concentrations (ng/mL) of Tigecycline in Monkeys After a
Single Intravenous Dose of 5 mg/kg

| Animal | | | | | | | | | | | | | |
|---|---|---|---|---|---|---|---|---|---|---|---|---|---|
| No. | Hours | 0 | 0.083 | 0.5 | 1 | 2 | 4 | 6 | 8 | 12 | 24 | 32 | 48 |
| 1 | | <100 | 15096 | 2030 | 1449 | 1228 | 721 | 517 | 429 | 264 | 167 | <100 | <100 |
| 2 | | <100 | 8136 | 1724 | 1449 | 1193 | 938 | 630 | 457 | 325 | 216 | 127 | 108 |
| 3 | | <100 | 14002 | 1890 | 1056 | 909 | 539 | 419 | 308 | 200 | 110 | <100 | <100 |
| 4 | | <100 | 23050 | 3340 | 1661 | 1013 | 588 | 431 | 372 | 265 | 155 | <100 | <100 |
| Mean | | 0 | 15071 | 2246 | 1404 | 1086 | 697 | 499 | 392 | 264 | 162 | 31.8 | 27.0 |
| SD | | 0 | 6135 | 740 | 252 | 151 | 178 | 97.5 | 66.0 | 51.0 | 43.6 | 63.5 | 54.0 |
| n | | 4 | 4 | 4 | 4 | 4 | 4 | 4 | 4 | 4 | 4 | 4 | 4 |

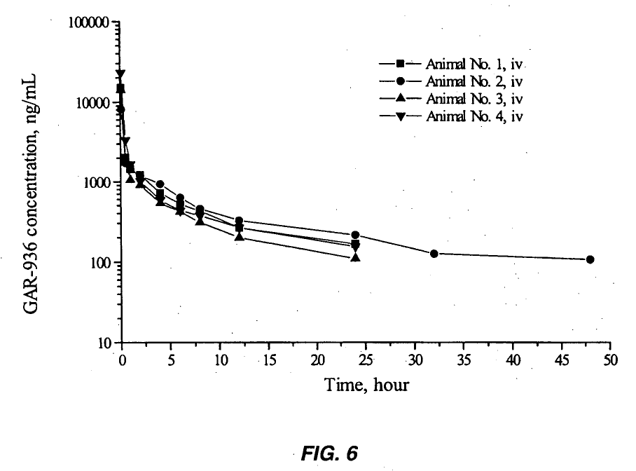

Plasma concentrations vs. time profiles after a single iv dose of tigecycline in monkeys are depicted in FIG. 6. Pharmacokinetic parameters from individual animals are tabulated in Table VII.

TABLE VII
Individual and Mean (±SD) Pharmacokinetic Parameters of Tigecycline
in Monkeys After a Single Oral (gavage) Dose of 15 mg/kg or After a Single
Intravenous Dose of 5 mg/kg

| Dose | | | Cₘₐₓ^{a} | tₘₐₓ | AUC₀₋ₜ | AUC0₋₄ | t_{1/2} | Cl_{T} | Vdₛₛ | MRTᵢᵥ |
|---|---|---|---|---|---|---|---|---|---|---|
| (mg/kg) | Route | Animal No. | (ng/mL) | (hr) | (ng · hr/mL) | (ng · hr/mL) | (hr) | (L/kg/hr) | (L/kg) | (hr) |
| 15 | oral | 1 | 131 | 2.0 | 151^{b} | nc | nc | NA | NA | NA |
| | | 2 | 191 | 2.0 | 242^{b} | nc | nc | NA | NA | NA |
| | | 3 | 178 | 1.0 | 105^{c} | nc | nc | NA | NA | NA |
| | | 4 | 150 | 2.0 | 154^{b} | nc | nc | NA | NA | NA |
| | | Mean | 163 | 1.8 | 163 | — | — | — | — | — |
| | | SD | 27.1 | 0.5 | 57.2 | — | — | — | — | — |
| | | n | 4 | 4 | 4 | | | | | |
| 5 | iv | 1 | 15096 | NA | NA | 18220 | 12.8 | 0.274 | 3.13 | 11.4 |
| | | 2 | 8136 | NA | NA | 20662 | 19.1 | 0.242 | 5.02 | 20.7 |
| | | 3 | 14002 | NA | NA | 14007 | 11.4 | 0.357 | 3.28 | 9.1 |
| | | 4 | 23050 | NA | NA | 20178 | 13.2 | 0.248 | 2.45 | 9.8 |
| | | Mean | 15071 | — | — | 18267 | 14.1 | 0.280 | 3.47 | 12.8 |
| | | SD | 6135 | — | — | 3030 | 3.4 | 0.053 | 1.09 | 5.4 |
| | | n | 4 | | | 4 | 4 | 4 | 4 | 4 |

^{a}Cₘₐₓ = C_{5min.} after the iv dose.
^{b}t = 2 hr for AUC determination.
^{c}t = 1 hr for AUC determination.
NA: Not applicable.
nc: AUC0₋₄ or t_{1/2} value not calculated due to insufficient data in the apparent terminal phase.

After a single 15 mg/kg oral (gavage) dose, tigecycline was detected in samples up to 2 hours post-dose. The mean (±SD) Cₘₐₓ value was 163±27.1 ng/mL and the tₘₐₓ values were between 1 and 2 hours. Due to the paucity of quantifiable concentrations in the terminal phase of the concentration vs. time curves after oral dosing, AUC₀₋₄,. and t_{1/2} values were not estimated after the oral dose. Also, due to the limited number of time points with quantifiable tigecycline concentration and the partial AUC values estimated, absolute bioavailability of tigecycline after oral dosing could not be determined.

A 0.5% blood bioavailability is suitable for treating GI tract infections since the desired site of action is in the GI tract and not in the blood. Thus, a 0.5% blood bioavailability can translate to approximately 99% bioavailability in the GI tract.

After a single 5 mg/kg intravenous dose in monkeys, the plasma concentrations of tigecycline declined polyexponentially. The mean t_{1/2} value estimated from the terminal phase of the plasma concentration vs. time curves was 14.1±3.4 hours, that was similar to the MRTᵢᵥ of 12.8±5.4 hours. The mean (±SD) AUC₀₋₄, value of tigecycline was 18267±3030 ng·hr/mL. The mean tigecycline Cl_{T} was 0.280±0.053 L/kg/hr and the mean Vdₛₛ was 3.47 ±1.09 L/kg.

Discussion.

The results of this study showed that the blood bioavailability of tigecycline was low after oral administration. Low blood bioavailability is desired because the drug is kept within the stomach for local action against the organisms in the GI tract. The absolute bioavailability could not be estimated after a single 15 mg/kg oral dose due to insufficient data in the terminal phase for the estimation of AUC₀₋₄ values. After a single iv dose in monkeys, the plasma concentrations of tigecycline declined polyexponentially. The terminal half-lives estimated from the terminal phase of the plasma concentration vs. time curves were between 11.4 and 19.1 (mean 14.1) hours and were similar to the MRTᵢᵥ (mean 12.8 hours). The systemic clearance (Cl_{T}) of GAR-93 6 in monkeys was relatively low (mean 0.280 L/kg/hr) but similar to that in dogs (ca. 0.26 L/kg/hr after a single 5 mg/kg dose). The steady-state volume of distribution (Vdₛₛ) of tigecycline in monkeys was large (3.47 L/kg) and in excess of the volume of total body water in this species (see Davies B, Morris T. “Physiological parameters in laboratory animals and humans.,” Pharm. Res. 1993; 10:1093-95), suggesting that tigecycline should be distributed to various tissues and organs.

### Example 3

This Example demonstrates the oral bioavailability in fasted male cynomolgus monkeys from an encapsulated microparticulate (100 mg) formulation administered as a single enteric coated oral formulation. Tigecycline plasma concentrations were determined for the formulation type by an LC/MS/MS method.

### Materials and Methods

Formulation

The tigecycline formulation was a 100 mg, encapsulated multi-particulate formulation having the components listed in Table VIII below:

TABLE VIII

| Granulation | % w/w | mg/250 mg |
|---|---|---|
| Tigecycline, 98% potency | 30.00 | 76.53 |
| Microcrystalline cellulose (Avicel PH1O1)^{a} | 22.00 | 53.47 |
| Mannitol DC grade | 30.00 | 75.00 |
| HPMC K100 (Dow) | 5.00 | 12.50 |
| Sodium Phosphate (dibasic) | 8.00 | 20.00 |
| Sodium stearyl fumarate (Pruv) | 1.50 | 3.75 |
| EDTA | 0.50 | 1.25 |
| Sodium starch glycolate | 3.00 | 7.50 |

^{a}Potency of tigecycline is adjusted against microcrystalline cellulose (MCC)

The enteric coating comprised a Seal Coat, YS-1-7006, and Enteric Coat (Acryl-EZE). The final potency for enteric coated tigecycline was 209 mg/g. Each 100 mg capsule contained 478.5 mg enteric coated granules.

Experimental Design and Sample Collection

The bioavailability of tigecycline was investigated with four male cynomolgus monkeys, each having body weights ranging from 5.5 to 7.1 kg. The monkeys were housed in Bioresources vivarium with free access to water and food. The four monkeys received the oral formulation described above (1×100 mg multi-particulate capsule). The formulation was administered with 10 mL water. All monkeys were fasted overnight prior to dosing (with free access to water) and were fed 4 hours after dose administration.

Blood samples were drawn from the saphenous vein at 0 (predose), 0.5, 1, 2, 3, 4, 8, 12 and 24 hours after dosing. Approximately 3 mL of blood were drawn into Vacutainer® tubes containing sodium heparin as the anticoagulant. Plasma was separated in a refrigerated centrifuge and stored at −70° C. Plasma samples were delivered to the assay site packed on dry ice.

Plasma tigecycline concentrations were determined by an LC/MS/MS method described above. Based on a 0.5 mL sample volume, the method has a limit of quantitation of 10 ng/mL.

Determination of Tigecycline Concentrations in Monkey Plasma

Tigecycline concentrations were determined by an LC/MS/MS method. Using 0.50 mL of sodium heparin monkey plasma, the lower limit of quantitation (LLOQ) was 10.0 ng/mL and the assay range was 10.0 to 1000 ng/mL. To monitor assay performance, all analytical runs were analyzed with low, mid-range, and high concentration (30, 300, and 800 ng/mL nominal concentrations) quality control samples (QCs) in quintuplets.

Analytical Performance of Tigecycline LC/MS/MS Assay in Monkey Plasma

There was one analytical run for the quantitation of tigecycline in monkey plasma samples from this study. The back-calculated values of tigecycline calibration standards prepared in monkey plasma and the calibration curve regression constants are shown in Table IX.

TABLE IX
Analytical Performance of Tigecycline Assay in Monkey Plasma:
Back-Calculated Concentrations of Calibration Standards and
Calibration Curve Regression Constants
(A) Back-Calculated Concentrations of Tigecycline Calibration
Standards in Monkey Plasma

| | Tigecycline Nominal Concentration, ng/mL | | | | | | | |
|---|---|---|---|---|---|---|---|---|
| Curve No. | 10 | 25 | 50 | 100 | 200 | 400 | 900 | 1000 |
| | Tigecycline Observed Concentration, ng/mL | | | | | | | |
| 1 | 9.72 | 25.3 | 51.9 | 113 | 221 | 384 | 796 | 895 |
| Mean | 9.72 | 25.3 | 51.9 | 113 | 221 | 384 | 796 | 895 |
| % Bias | −2.8 | 1.2 | 3.8 | 13.0 | 10.5 | −4.0 | −11.6 | −10.5 |
| n | 1 | 1 | 1 | 1 | 1 | 1 | 1 | 1 |

(B) Calibration Curve^{a} Regression Constants for Tigecycline
Assay in Monkey Plasma

| | Curve No. | Slope | Intercept | R² |
|---|---|---|---|---|
| | 1 | 0.00190 | 0.00917 | 0.9895 |
| Mean | | 0.00190 | 0.00917 | 0.9895 |
| n | | 1 | 1 | 1 |

^{a}A linear regression method was used with 1/concentration² as the weighting factor.

Linear regression was performed using a weighting factor of 1/(concentration)². The mean biases of back-calculated calibration standards ranged from −11.6% to 13.0%. The R² value of the calibration curve was 0.9895.

Results of tigecycline quality control (QC) samples prepared in monkey plasma and analyzed with the study samples are summarized in Table X.

TABLE X
Analytical Performance of Tigecycline Assay in Monkey Plasma:
Results of Quality Control (QC) SamDles
Tigecycline QC Samples

| | Curve | Low QC | Middle QC | High QC |
|---|---|---|---|---|
| | Number | (30 ng/mL) | (300 ng/mL) | (800 ng/mL) |
| | 1 | 28.1 | 279 | 702 |
| | | 27.3 | 277 | 682 |
| | | 28.6 | 261 | 690 |
| | | 30.1 | 302 | 666 |
| | | 31.8 | 296 | 691 |
| Mean | | 29.2 | 283 | 686 |
| S.D. | | 1.79 | 16.3 | 13.3 |
| % CV | | 6.1 | 5.8 | 1.9 |
| % Bias | | −2.7 | −5.7 | −14.3 |
| n | | 5 | 5 | 5 |

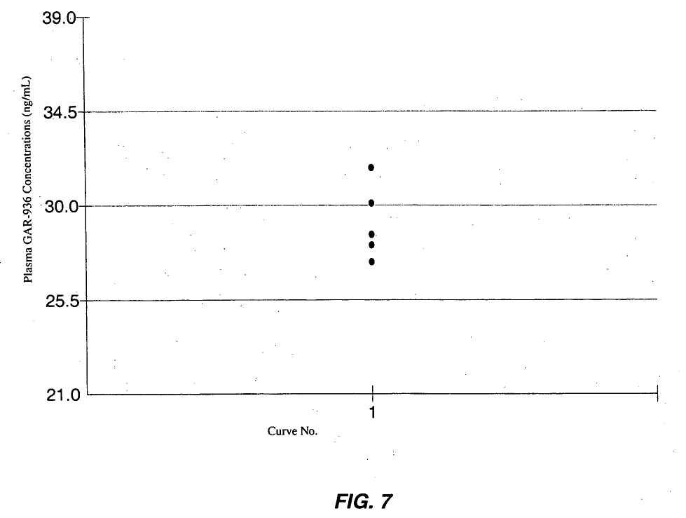

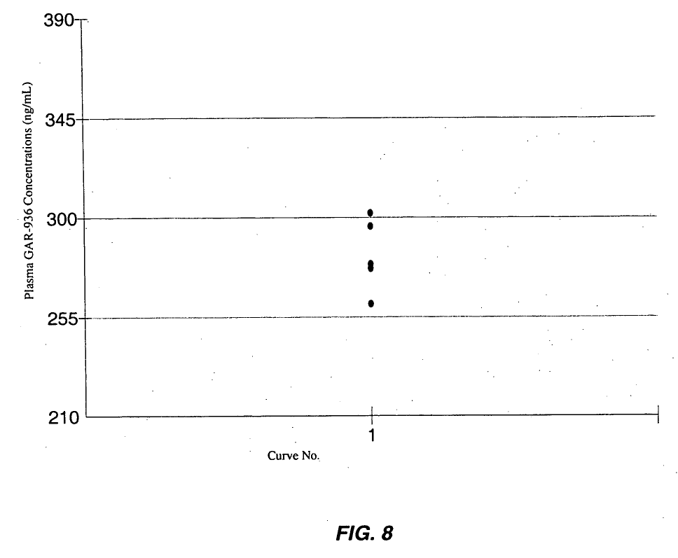

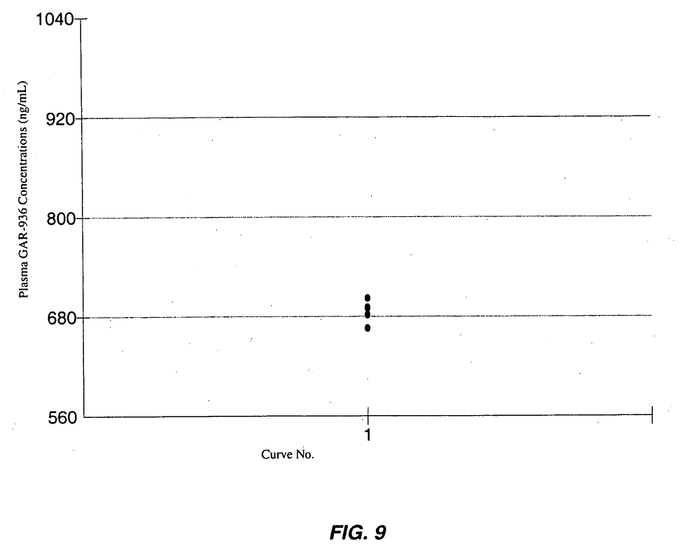

The CV of the QC samples ranged from 1.9% to 6.1% and the mean biases ranged from −14.3% to −2.7%. The QC results are also depicted graphically in FIGS. 7 to 9.

Plasma Concentrations of Tigecycline in Monkeys

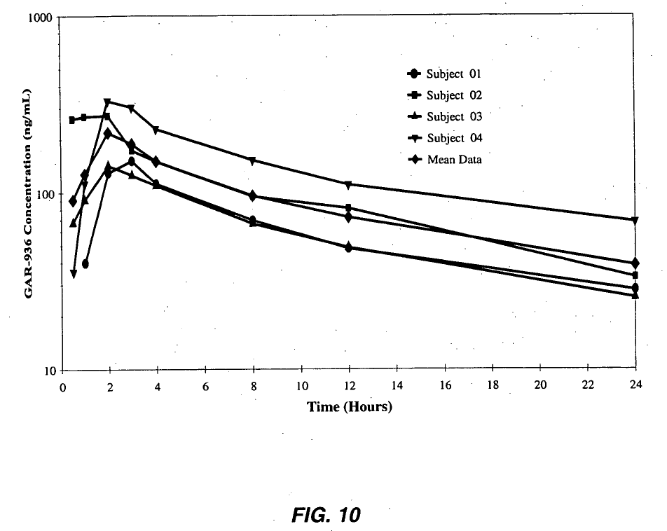

Tigecycline plasma concentrations (ng/mL) in fasted monkeys after a single oral dose (100 mg capsule) of tigecycline from an encapsulated microparticulate formulation are presented in Table XI and shown graphically in FIG. 10.

TABLE XI
Plasma Concentrations (ng/mL) of Tigecycline After A Single
Oral Dose (100 mg Tigecycline Encapsulated Microparticulate
Capsule) in Fasted Male Cynomolcus Monkeys

| SAN* | 0 hr | 0.5 hr | 1 hr | 2 hr | 3 hr | 4 hr | 8 hr | 12 hr | 24 hr |
|---|---|---|---|---|---|---|---|---|---|
| | Tigecycline Concentration, ng/mL | | | | | | | | |
| 1 | <10.0 | <10.0 | 39.9 | 130 | 152 | 113 | 69.6 | 48.1 | 28.1 |
| 2 | <10.0 | 261 | 270 | 273 | 174 | 151 | 95.3 | 81.6 | 33.1 |
| 3 | <10.0 | 67.4 | 90.9 | 143 | 126 | 110 | 66.6 | 48.8 | 25.4 |
| 4 | <10.0 | 35.6 | 113 | 331 | 304 | 230 | 153 | 111 | 68.2 |
| Mean | 0 | 91.0 | 128 | 219 | 189 | 151 | 96.1 | 72.4 | 38.7 |
| SD | 0 | 117 | 99.2 | 98.6 | 79.1 | 55.9 | 40.0 | 30.1 | 19.9 |
| % CV | 0 | 128.6 | 77.5 | 45.0 | 41.9 | 37.0 | 41.6 | 41.6 | 51.4 |
| n | 4 | 4 | 4 | 4 | 4 | 4 | 4 | 4 | 4 |

*SAN: Study animal number

Plasma Concentration-Time Data Analysis

Noncompartmental analysis of the individual monkey plasma tigecycline concentration-time profiles was performed using WinNonlin, Model 200. Area under the plasma tigecycline concentration-time curves (AUC) were calculated by log/linear trapezoid rule. The peak plasma tigecycline concentrations (Cₘₐₓ) and the time to reach Cₘₐₓ (tₘₐₓ) were noted directly from the plasma tigecycline concentration-time profiles.

The AUC (ng·hr/mL, mean±SD) value for the formulation was 2830±1111. The Cₘₐₓ value (ng/mL, mean±SD) for the formulation was 225±92.4.

Pharmacokinetics

The individual and mean monkey pharmacokinetic parameters are reported in Table XII.

TABLE XII
Individual and Mean Pharmacokinetic Parameters of Tigecycline After
A Single Dose (100 mg Encapsulated Microparticulate Capsule. Batch L23290-
29B) in Fasted Male Cynomolgus Monkeys

| | | | | AUC_{0/24} | AUC_{0-∞} | | | |
|---|---|---|---|---|---|---|---|---|
| Monkey | Dose | Cmax | Tmax | (ng | (ng | T1/2 | | |
| SAN | (mg/kg) | (ng/mL) | (hr) | hr/mL) | hr/mL) | (hr) | AUC/Dose | Cmax/Dose |
| 01 | 14.1 | 152 | 3.0 | 1430 | 1950 | 12.8 | 138 | 10.8 |
| 02 | 14.9 | 273 | 2.0 | 2390 | 2840 | 9.48 | 191 | 18.3 |
| 03 | 16.7 | 143 | 2.0 | 1460 | 1890 | 11.8 | 113 | 8.56 |
| 04 | 18.2 | 331 | 2.0 | 3220 | 4640 | 14.4 | 255 | 18.2 |
| Mean | 16.0 | 225 | 2.25 | 2130 | 2830 | 12.1 | 174 | 14.0 |
| S.D. | 1.83 | 92.4 | 0.5 | 855 | 1111 | 2.06 | 62.7 | 5.04 |
| % GV | 11.4 | 41.1 | 22.2 | 40.2 | 39.2 | 17 | 36.0 | 36.1 |
| n | 4 | 4 | 4 | 4 | 4 | 4 | 4 | 4 |

Table XIII compares the mean pharmacokinetic parameters and the absolute and relative bioavailability of tigecycline in the encapsulated multi-particulate formulation to the 0.9% saline tigecycline solution administered IV and orally (gavage), as described in Example 2 above.

TABLE XIII
Comparison of Pharmacokinetic Parameters [Mean (n = 4)]
in Male Cynomolgus Monkeys After A Single Dose
Administration of Tigecycline

| | | 15 mg/kg | |
|---|---|---|---|
| | 16 mg/kg 100 | 0.9% saline, | 15 mg/kg |
| Parameter | mg oral capsule | Gavage¹ | IV Gavage |
| AUC_{0/t or 0-∞} | 2830 | 163 | 18267 |
| AUC/Dose | 174 | 10.9 | 3653 |
| Cmax (ng/mL) | 225 | 163 | 15071 |
| Cmax/Dose | 14.0 | 10.9 | 3014 |
| tmax (hr) | 2.25 | 1.8 | Not applicable |
| t1/2(hr) | 12.1 | Not calculated | 14.1 |
| Bioavailability | 4.8% | — | — |

¹See Example 2

The AUC (ng·hr/mL, mean±SD) value for the formulation was 2830±1111. The Cₘₐₓ values (ng/mL, mean±SD) for the formulation was 225±92.4.

A bioavailability study of a tigecycline formulation has been conducted in cynomolgus monkeys to assess the bioavailability of an enhanced encapsulated microparticulate oral dosage formulation.

The results of this study showed that the absolute bioavailability of tigecycline in the blood was 5% after oral administration. The capsule formulation (16 mg/kg) demonstrated significantly higher oral exposure (AUC) values as compared to previous studies conducted at 15 mg/kg. Thus, 95% of the drug is present in the GI tract.

### Example 4

This Example describes a dry powder layering process for the preparation of an oral formulation. Table XIV lists the formulation ingredients.

TABLE XIV

| Ingredient | % w/w | mg/250 mg |
|---|---|---|
| Tigecycline (98% | 60.0 | 150.00 |
| potency) | | |
| lactose | 31.5 | 78.75 |
| Sodium phosphate | 5.0 | 12.5 |
| (dibasic) | | |
| EDTA | 0.5 | 1.25 |
| Hypromellose solution | 5-10% solution | |
| Enteric Coat (Acryl- | 10-30% weight | |
| EZE), 93018429 | gain on dry | |
| | layered pellets | |

In this example the tigecycline, lactose, sodium phosphate and EDTA were blended together and fed through a screw feed into a fluid bed rotor granulator containing sucrose or microcrystalline spheroids. A 5-10% binder solution of hypromellose was sprayed simultaneously into the spinning bed of spheroids while the tigecycline blend was slowly added. After the desired quantity of tigecycline blend was added to the spheres, they were dried and discharged for enteric coating. Enteric coating was applied via a fluid bed processor using polymethacrylates. Other enteric polymers normally used in industry can also be used.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

What is claimed is:

1. A method of treating at least one bacterial infection, comprising:
orally administering to a subject in need thereof a pharmaceutical composition comprising a therapeutically effective amount of tigecycline.

2. The method according to claim 1, wherein the at least one bacterial infection is a gastrointestinal infection.

3. The method according to claim 1, wherein the at least one bacterial infection is a lower gastrointestinal tract infection.

4. The method according to claim 1, wherein the at least one bacterial infection is caused by anaerobic bacteria.

5. The method according to claim 1, wherein the at least one bacterial infection is caused by Clostridium difficile.

6. The method according to claim 1, wherein the pharmaceutical composition is in liquid form.

7. The method according to claim 6, wherein the liquid form comprises a solution or suspension.

8. The method according to claim 7, wherein the solution or suspension has a pH less than 7.5.

9. The method according to claim 6, wherein the pharmaceutical composition is a saline solution containing tigecycline.

10. The method according to claim 6, wherein the composition is a suspension comprising tigecycline.

11. The method according to claim 1, wherein the pharmaceutical composition is in solid form.

12. The method according to claim 11, wherein the solid form is chosen from tablets, capsules, powders, and lyophilized cakes and powders.

13. The method according to claim 11, wherein the pharmaceutical composition comprises tigecycline having at least one enteric coating.

14. The method according to claim 13, wherein the tigecycline is multi-particulate.

15. The method according to claim 13, wherein the at least one enteric coating is chosen from dimethylaminoethyl methacrylatemethylacrylate acid ester copolymer, anionic acrylic resins such as methacrylic acid/methyl acrylate copolymer and methacrylic acid/ethyl acrylate copolymer, ethylacrylate-methylmethacrylate copolymer, hydroxypropylmethylcellulose acetate succinate (HPMCAS), hydroxypropylmethylcellulose phthalate (HPMCP), cellulose acetate phthalate (CAP), carboxymethylcellulose acetate phthalate (CMCAP), hydroxypropylmethylcellulose, hydroxyethylcellulose, methylhydroxyethylcellulose, sodium carboxymethylcellulose, hydroxypropylcellulose, polyvinyl pyrrolidone, shellac, methylcellulose, and ethylcellulose, and blends and copolymers thereof.

16. The method according to claim 11, wherein the oral dosage form is chosen from capsules, tablets, pills, powders, and granules.

17. The method according to claim 11, further comprising at least one base.

18. The method according to claim 17, wherein the at least one base is chosen from phosphates, carbonates, bicarbonates, citrates, and tartrates.

19. The method according to claim 11, further comprising at least one chelating agent.

20. The method according to claim 19, wherein the at least one chelating agent is chosen from EDTA, EGTA, tartrates, and citrates.

21. The method according to claim 11, further comprising at least one biopolymer.

22. The method according to claim 21, wherein the at least one biopolymer is chosen from hypromellose, xanthan gum, and carbomer.

23. The method according to claim 11, further comprising at least one base, at least one chelating agent, and at least one biopolymer.

24. The method according to claim 1, wherein the pharmaceutical composition comprises enteric coated multi-particulate pellets incorporated into a hard gelatin capsule, each pellet comprising tigecycline and microcrystalline cellulose, and at least one component chosen from at least one base, at least one chelating agent, and at least one biopolymer.

25. The method according to claim 1, wherein the pharmaceutical composition comprises tigecycline and microcrystalline cellulose, and further comprising at least one component chosen from at least one base, at least one chelating agent, and at least one biopolymer.

26. The method according to claim 1, wherein the pharmaceutical composition comprises multi-particulate pellets incorporated into an enteric coated soft gelatin capsule, each pellet comprising tigecycline and microcrystalline cellulose, and further comprising at least one component chosen from at least one base, at least one chelating agent, and at least one biopolymer.

27. The method according to claim 1, wherein the pharmaceutical composition comprises an enteric coated soft liquid gel capsule, and further comprising a non-aqueous solution of tigecycline and at least one component chosen from at least one base, at least one chelating agent, and at least one biopolymer.

28. The method according to claim 1, wherein the tigecycline is in solid form, and the pharmaceutical composition further comprises lactose and at least one acidifying agent.

29. The method according to claim 28, wherein the acidifying agent is HCl.

30. The method according to claim 1, wherein the orally administering comprises administering through a nasal gastric tube.

31. The method according to claim 1, wherein the pharmaceutical composition comprises a suspension, wherein the suspension comprises granules and at least one suspending agent.

32. The method according to claim 31, wherein the at least one suspending agent is chosen from xanthan gum, guar gum, gum arabic, and hydroxypropylmethylcellulose.

33. A method of treating antibiotic associated pseudomembranous colitis caused by C. difficile, and enterocolitis caused by S. aureus and associated methicillin resistant strains comprising:
orally administering to a subject in need thereof a pharmaceutical composition comprising a therapeutically effective amount of tigecycline.
